(19) United States
(12) Patent Application Publication
(10) Pub. No.: US 2008/0177367 A1
(43) Pub. Date: Jul. 24, 2008

(54) Treatment device for treating a side branch of a vessel and method for positioning a stent in a side branch

(76) Inventor: Adam Stys, Sioux Falls, SD (US)

Correspondence Address: Roeder & Broder LLP, 5560 Chelsea Avenue, La Jolla, CA, 92037, US

(21) Appl. No.: 11/656,242

(22) Filed: Jan. 22, 2007

(51) Int. Cl. A61F 2/84

(52) U.S. Cl. 623/1.11

(57) ABSTRACT

A treatment device (16C) for treating a treatment site (14) of a vessel (12) includes a tubular shaft (430), a first passageway (434), and a second passageway (436). The shaft (430) includes a distal shaft region (430A) that is sized and shaped to fit within the vessel (12) and a proximal shaft region (430B) that is used to move the distal shaft region (430A) in the vessel (12). The first passageway (434) receives a first guide wire (16A) and extends along at least a portion of the shaft (430). The first passageway (434) includes a first passageway inlet (434A) that receives the first guide wire (16A) and a first passageway outlet (434B). The first passageway inlet (434A) is positioned between the distal shaft region (430A) and the proximal shaft region (430B). The first passageway outlet (434B) is positioned near the proximal region (430B). The second passageway (436) receives a second guide wire (16B) and extends along the shaft (430). The second passageway (436) includes a second passageway inlet (436A) that receives the second guide wire (16B) and a second passageway outlet (436B). The second passageway inlet (436A) is positioned at the distal shaft region (430A), and the second passageway outlet (436B) is positioned near the proximal shaft region (430B). With this design, the two guide wires (16A), (16B) and the treatment device (16C) can be used to position a stent (18) at a treatment site (14) that is located at an intersection of an angled side branch (12B) and a main branch (12A) of the vessel (12).

### BACKGROUND

The process of atherosclerosis causes fatty deposits (plaque) to accumulate in the walls of arteries. As the process becomes more advanced, the fatty deposits begin to encroach on the lumen of the artery, resulting in blockages (stenosis) of varying degrees and reduction in blood flow. One treatment of such blockages is a procedure commonly referred to as angioplasty. In a typical angioplasty procedure, a balloon is used to open the blockage at a treatment site to restore the blood flow. In certain patients, the artery begins to renarrow at the treatment site shortly after the angioplasty procedure. This is referred to as restenosis. As a result thereof, another angioplasty procedure many be necessary at the treatment site within a few months.

One method used to reduce restenosis includes positioning a stent in the artery at the treatment site. A stent is a tubular structure that is inserted into the artery and subsequently expanded against the inner wall of the artery at the treatment site to support the inner wall. In certain patients, the stent reduces the rate of renarrowing at the treatment site.

A typical artery can include a main branch and a plurality of side branches that extend away from the main branch. These side branches are at a variety of angles relative to the main branch. Unfortunately, because of the geometry at an intersection of the side branch and the main branch, existing stents do not adequately support a treatment site that is located near the intersection. As a result thereof, treatment sites located near the intersection often suffer from relatively frequent restenosis. Further, existing balloon catheters are not able to accurately deliver a stent to a treatment site located near the intersection.

### SUMMARY

The present invention is directed to a treatment device for treating a treatment site of a vessel of a mammal. The treatment device includes a tubular shaft, a first passageway, and a second passageway. The shaft includes a distal shaft region that is sized and shaped to fit within the vessel and a proximal shaft region that is used to move the distal shaft region in the vessel. The first passageway receives a first guide wire and extends along at least a portion of the shaft. The first passageway includes a first passageway inlet that receives the first guide wire and a first passageway outlet. The first passageway inlet is positioned between the distal shaft region and the proximal shaft region. The first passageway outlet is positioned near the proximal region. The second passageway receives a second guide wire and extends along the shaft. The second passageway includes a second passageway inlet that receives the second guide wire and a second passageway outlet. The second passageway inlet is positioned at the distal shaft region, and the second passageway outlet is positioned near the proximal region. With this design, the treatment device is particularly useful for positioning and properly orienting a stent at a treatment site located near an ostium of a side branch of the vessel. This increases the likelihood that the procedure performed on the patient will be successful, by precisely positioning a stent proximal edge at the ostium.

In one embodiment, the shaft section includes a first lumen that defines at least a portion of the first passageway, and a second lumen that defines the second passageway.

Additionally, the treatment device can include an expander that is secured to the shaft near the distal shaft region. The expander is movable between a retracted position and an expanded position and includes a distal expander edge and a proximal expander edge. For example, the expander can be an inflatable balloon.

In one embodiment, the first passageway inlet is positioned closer to the proximal expander edge than the distal expander edge. For example, the first passageway can extend through at least a portion of the expander and the first passageway inlet can be positioned in the expander. Alternatively, the first passageway inlet can be positioned in the shaft proximal to the expander.

The present invention is also directed to a method for treating a treatment site of a vessel of a mammal. The vessel includes a main branch, and a side branch. The treatment site is located in the side branch near an intersection of the side branch and the main branch. The method includes the steps of: (i) providing a first guide wire having a first distal end and a first proximal end; (ii) moving the first distal end of the first guide wire in the main branch until the first distal end is past the intersection of the side branch with first distal end still in the main branch; (iii) providing a second guide wire having a second distal end and a second proximal end; (iv) moving the second distal end of the second guide wire in the main branch and into side branch until the second distal end is past the treatment site; (v) providing a treatment device including (i) a tubular shaft having a distal shaft region that is sized and shaped to fit within the vessel and a proximal shaft region that is used to move the distal shaft region in the vessel; (ii) a first passageway that extends along at least a portion of the shaft and that receives the first guide wire, the first passageway having a first passageway inlet that receives the first guide wire and a first passageway outlet, the first passageway inlet being positioned between the distal shaft region and the proximal shaft region, and the first passageway outlet being positioned near the proximal region; and (iii) a second passageway that extends along the shaft and that receives the second guide wire, the second passageway having a second passageway inlet that receives the second guide wire and a second passageway outlet, the second passageway inlet being positioned at the distal shaft region, and the second passageway outlet being positioned near the proximal region; and (vi) moving the treatment device over the guide wires until a portion of the treatment device is positioned adjacent to the treatment site.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified, cut-away, side illustration of a portion of a vessel, a device positioner, and a stent in an expanded configuration positioned in the vessel;

FIG. 2A is a simplified side view of the stent of FIG. 1 in the expanded configuration and a cut-away view of a portion of the vessel;

FIG. 2B is a simplified side view of the stent of FIG. 1 in a retracted configuration;

FIG. 2C is a simplified perspective view of the stent of FIG. 1 in the expanded configuration;

FIG. 3 is a simplified side view of a plurality of stents having features of the present invention;

FIG. 4 is a simplified, cut-away, side illustration of a portion of the vessel, the device positioner, and the stent in a folded configuration positioned in the vessel;

FIG. 5 is a simplified, cut-away view of a portion of the treatment device; and

FIG. 6 is a simplified, cut-away view of a portion of another embodiment of the treatment device.

### DESCRIPTION

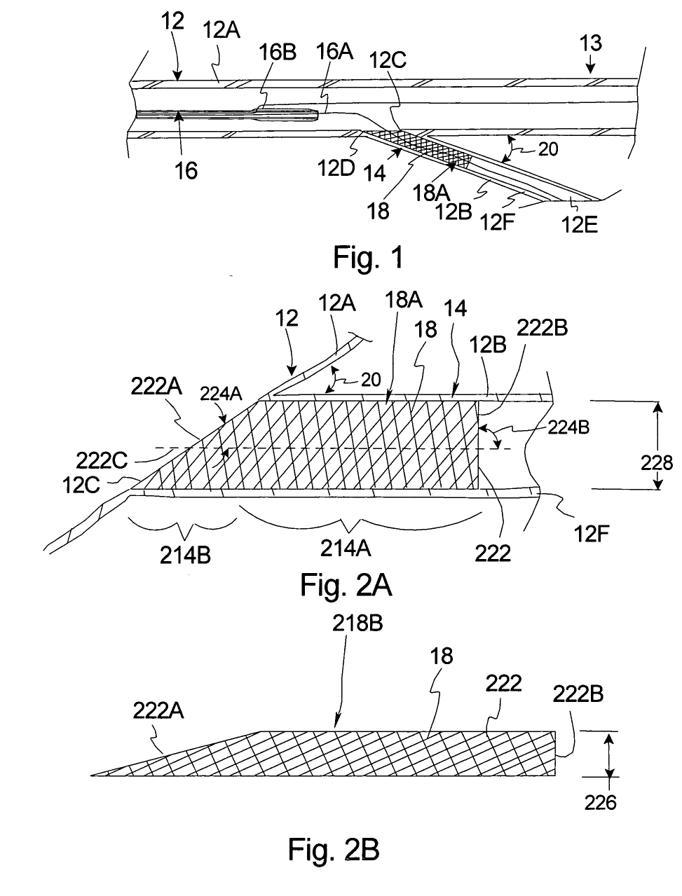

FIG. 1 is a simplified, cut-away side illustration of a portion of a vessel 12 of a patient 13 having a treatment site 14, a stent delivery device 16, and a stent 18 positioned at the treatment site 14 with the stent delivery device 16. In FIG. 1, the vessel 12 includes a main branch 12A and a side branch 12B. As an overview, in certain embodiments, the stent 18 is uniquely designed to fully support an ostium 12C of the side branch 12B. Stated in another fashion, in certain embodiments, the stent 18 is uniquely designed to fully support the entire treatment site 14 when the treatment site 14 is located near an intersection 12D of the side branch 12B and the main branch 12A. This increases the likelihood that the treatment site 14 will be held open, reduces the likelihood of restenosis at the treatment site 14, and increases the likelihood that the procedure performed on the patient 13 will be successful.

Further, in certain embodiments, the stent delivery device 16 is uniquely designed to properly position and properly orient the stent 18 at the treatment site 14. This simplifies the placement of the stent 18 and increases the likelihood that the procedure performed on the patient 13 will be successful when it comes to the precision of the deployment.

The type of vessel 12 and treatment site 14 can vary. For example, the vessel 12 can be an artery of a mammal, such as a human being. Alternatively, for example, the vessel 12 can be another body passageway in the vascular system or an organ. In FIG. 1, the main branch 12A and the side branch 12B each include a vessel lumen 12E and a vessel wall 12F. The location of the side branch 12B relative to the main branch 12A can vary. In certain embodiments, the side branch 12B is at a branch angle 20 that is other than ninety degrees relative to the main branch 12A and the stent is designed to accommodate these angles ensuring full coverage/support of the ostium. In alternative, non-exclusive embodiments, the branch angle 20 is approximately less than approximately 70, 65, 60, 55, 50, 45, 40, 35, 30, or 25 degrees.

Further, in FIG. 1, the treatment site 14 is in the side branch 12B near the ostium 12C of the side branch 12B, and near the intersection 12D of the main branch 12 and the side branch 12B. In one embodiment, the treatment site 14 includes a fatty deposit of material (not shown), e.g. plaque, on the inner lining of the vessel wall 12F. In this embodiment, the stent 18 is used to open and/or hold open the vessel lumen 12E at the treatment site 14. Alternatively, for example, the stent 18 can be used to treat another type of problem with the vessel 12.

The stent delivery device 16 is used by the physician to position the stent 18 at the treatment site 14 in the vessel 12. FIG. 1 illustrates the stent delivery device 16 after the stent 18 has been deployed at the treatment site 14. The type of stent delivery device 16 utilized can be varied to suit the size and configuration of the vessel 12. In FIG. 1, the stent delivery device 16 includes a first guide wire 16A that is inserted into the main branch 12A past the ostium 12C of the side branch 12B, a second guide wire 16B that is inserted into the main branch 12A and into the side branch 12B past the treatment site 14, and a treatment device 16C, e.g. a balloon catheter that is guided to the treatment site 14 over the guide wires 16A, 16B. In this embodiment, as described in more detail below, the two guide wires 16A, 16B cooperate to (i) guide the treatment device 16C to the treatment site 14, (ii) properly position the treatment device 16C at the treatment site 14, and (iii) properly orientate the stent 18 at the treatment site 14. This simplifies the placement of the stent 18 and increases the likelihood that the procedure performed on the patient 13 will be successful.

The guide wires 16A, 16B as well as the treatment device 16C and can be introduced into the vessel 12 wherever it is most convenient to do so.

In certain embodiments, placement of the stent 18 is preceded by an angioplasty procedure that predilates the treatment site 14 and makes it easier to position the stent 18. A balloon catheter somewhat similar to the treatment device 16C illustrated in FIG. 1 can be used to dilate the treatment site 14 prior to positioning the stent 18. Alternatively, the stent 18 can be placed without a prior angioplasty procedure.

FIG. 1 illustrates the stent 18 in an expanded configuration 18A after it has been deployed at the treatment site 14 in the side branch 12B. FIG. 1, also illustrates that the stent 18 fully supports the treatment site 14 near the ostium 12C and the intersection 12D. This increases the likelihood that the entire treatment site 14 will be held open and increases the likelihood that the procedure performed on the patient 13 will be successful.

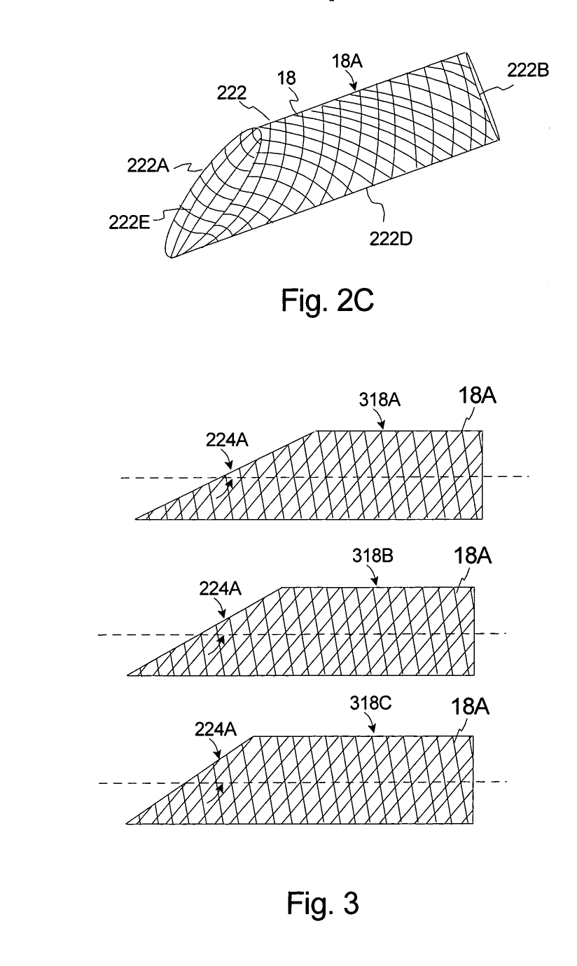

FIG. 2A is a side view of the stent 18 in the expanded configuration 18A and a portion of the vessel 12, FIG. 2B is a side view of the stent 18 in a retracted (“folded”) configuration 218B, and FIG. 2C is a perspective view of the stent 18 in the expanded configuration 18A. In this embodiment, the stent 18 includes a stent frame 222 that is moveable from the retracted configuration 218B prior to positioning at the treatment site 14 to the expanded configuration 18A in which the stent frame 222 supports the vessel wall 12F. In this embodiment, the stent frame 222 is tubular shaped and includes a first edge 222A, an opposed second edge 222B, a longitudinal axis 222C, an outer circumference 222D, and an inner circumference 222E. In one embodiment, when the stent 18 is positioned at the treatment site 14, the first edge 222A is positioned near the ostium 12C, and the second edge 222B is positioned away from the ostium 12C.

In certain embodiments, in the expanded configuration 18A, the tubular first edge 222A is at a first edge angle 224A relative to the longitudinal axis 222C that is less than ninety degrees relative to the longitudinal axis 222C. As a result thereof, the expanded stent frame 222 is better suited to support the entire treatment site 14 of the side branch 12B near the ostium 12C. In alternative, non-exclusive embodiments, the first edge angle 224A is approximately 70, 65, 60, 55, 50, 45, 40, 35, 30, or 25 degrees. Further, in certain embodiments, in the expanded configuration 18A, the first edge angle 224A is approximately equal to the branch angle 20. For example, in alternative, non-exclusive embodiments, in the expanded configuration 18A, the first edge angle 224A is within approximately 30, 25, 20, 15, 10, or 5 degrees of the branch angle 20. Generally, the stent 18 will better support the treatment site 14 as the first edge angle 224A approaches the branch angle 20.

FIG. 2A illustrates the treatment site 14 in the side branch 12B near the intersection 12D. Because of this location, the treatment site 14 can be divided to include a tubular, somewhat cylindrical shaped region 214A, and a tubular, somewhat triangular shaped region 214B. Further, because, in the expanded configuration 18A, the first edge 222A is at the first edge angle 224A, the stent 18 can fully support the triangular shaped region 214A.

Additionally, in the expanded configuration 18A, the second edge 222B is at a second edge angle 224B relative to the longitudinal axis 222C. In certain embodiments, the second edge angle 224B is less critical to the successful use of the stent 18. In FIG. 2A, the second edge angle 224 is approximately ninety degrees.

The stent frame 222 may be fabricated in a large range of diameters and lengths. In the retracted configuration 218B, the stent frame 222 has an outer diameter 226 which is less than the inner diameter of the vessel lumen 12E. With this design, in the retracted configuration 218B, the stent 18 can be moved in the vessel 12 to the treatment site 14.

Alternatively, in the expanded configuration 18A, the stent frame 222 has an outer diameter 228 that is approximately equal to or slightly greater than the desired diameter of the vessel 12 at the treatment site 14. With this design, the stent 18 can be placed and retained at the treatment site 14.

Further, the length of the stent frame 222 can be varied to suit the length of the treatment site 14. In non-exclusive examples, the stent frame 222 in the expanded configuration 18A may range in length from about eight millimeters to forty millimeters (8.0 mm-40.0 mm) and have an outer diameter 228 of between approximately 2 and 40 millimeters. However, the stent frame 222 can have other lengths and/or diameters.

In the embodiment illustrated in the Figures, the stent frame 222, for example, is a wire mesh. The invention is not intended to describe the exact stent struts structure as any stent 18 can be used with this concept. The invention describes the new stent 18 shape for the full coverage of the ostium 12C of the side branch 12B and a method of delivery that ensures that the stent 18 is properly positioned. In this embodiment, during movement of the stent frame 222 from the retracted configuration 218B to the expanded configuration 18A, the diameter of the stent frame 222 increases significantly and the length of the stent frame 222 decreases minimally. While a wire mesh construction is illustrated in the Figures, it should be understood that any other constructions may also be employed without departing from the spirit and scope of the invention. For example, in an alternative embodiment, the stent frame 222 can include a series of separate tubular shaped bands that are interconnected by one or more elongated strips, a helical coil, or another tubular structure that includes a plurality of apertures. Again, in certain embodiments, the spirit and scope of the invention encompasses the angled shape of the first edge angle 224A and the method of delivery of the stent 18 to precisely align the angle 224A with the angle 20 of the ostium 12C.

The stent frame 222 can be made of stainless steel, a shape memory material, or another suitable material.

In one embodiment, the stent frame 222 is moved from the retracted configuration 218B to the expanded configuration 18A with the balloon catheter 16C (illustrated in FIG. 1). Alternatively, for example, the stent frame 222 can be initially retained in the retracted configuration 218B with a retainer (not shown), e.g. an adhesive or a tubular sheath, and subsequently, when the retainer is removed, the stent frame 222 is self expanding and moves from the retracted configuration 218B to the expanded configuration 18A.

In one embodiment, in addition to supporting the vessel 12, the stent 18 also delivers a treatment (not shown), e.g. local drug delivery, to the vessel 12 at the treatment site 14. Stated another way, the stent frame 222 can emit and/or deliver a treatment to the treatment site 14. For example, the stent frame 222 can be coated with one or more treatments, e.g. drugs or therapeutic agents or molecules that have beneficial effects on the treatment site 14. The treatments may be bound to the stent frame 222 directly or with a polymer. Alternatively, the polymer coating of the stent frame 222 can consist of therapeutic molecules that are released at the treatment site 14 as the polymer degrades. Alternatively, the material of the stent frame 222 can emit the treatment.

Alternatively, the stent frame 222 can be entirely biodegradable, dissolving over a period of time after or coincident with the delivery of a treatment.

The design of the treatment can depend upon the treatment site 14. For example, the treatment can prevent plaque rupture, stabilize vulnerable plaque, cause a reduction in plaque volume, or inhibit new plaque development.

FIG. 3 is a simplified side view of a combination that includes three alternative stents 318A, 318B, 318C in the expanded configuration 18A. In this embodiment, each of the stents 318A, 318B, 318C has a different first edge angle 224A. With this design, the physician can evaluate the treatment site 14 (illustrated in FIG. 1) and use the stent 318A, 318B, 318C that will provide the best coverage for the treatment site 14. It should be noted that he physician can have more than three or fewer than three different stents 318A, 318B, 318C to select from.

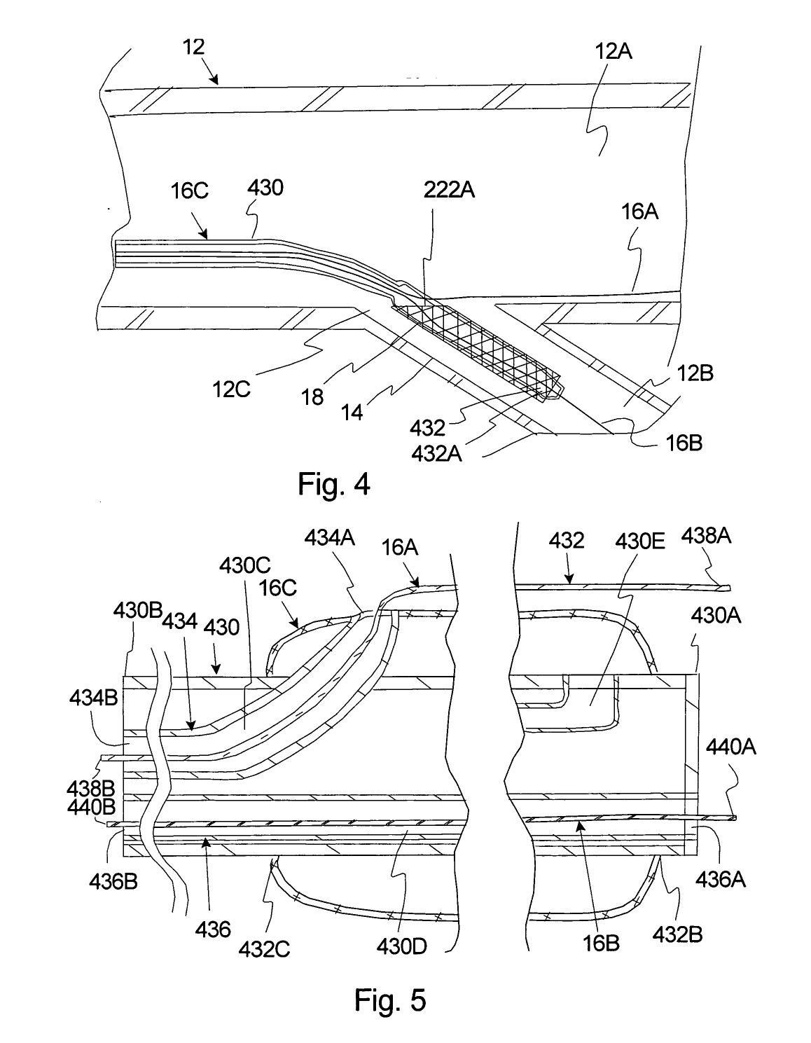

FIG. 4 illustrates the treatment device 16C positioned adjacent the treatment site 14 with the stent 18 positioned on the treatment device 16C prior to the stent 18 being deployed at the treatment site 14. Further, FIG. 4, illustrates (i) the first guide wire 16A has been inserted into the main branch 12A past the ostium 12C, and (ii) the second guide wire 16B has been inserted into vessel 12, and into the side branch 12B past the ostium 12C of the side branch 12B and past the treatment site 14.

FIG. 4 illustrates that with the unique design of the treatment device 16C, the two guide wires 16A, 16B cooperate to (i) guide the treatment device 16C to the treatment site 14, (ii) properly position the treatment device 16C at the treatment site 14, and (iii) properly orientate the stent 18 at the treatment site 14. This simplifies the placement of the stent 18 and increases the likelihood that the procedure performed on the patient 13 will be successful.

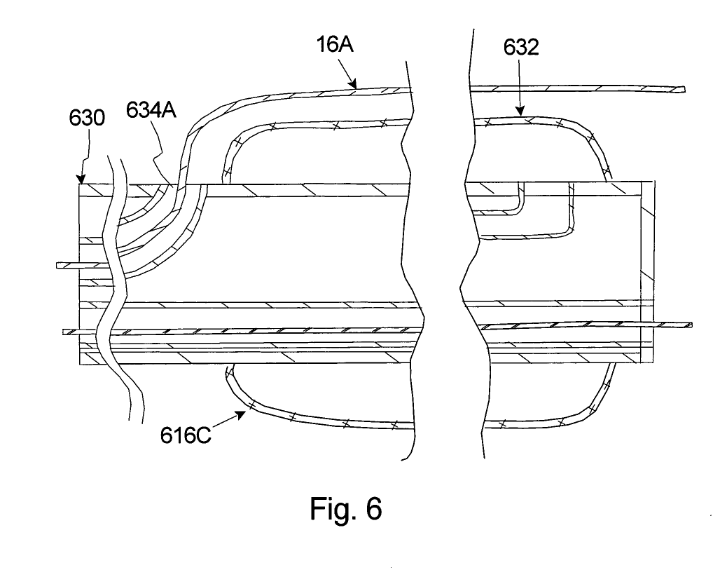

FIG. 5 illustrates an enlarged view of a portion of treatment device 16C. Referring to both FIGS. 4 and 5, in this embodiment, the treatment device 16C includes a tubular shaft 430, an expander 432, a first passageway 434, and a second passageway 436. The design of each of these components can be varied pursuant to the teachings provided herein.

The tubular shaft 430 includes a distal shaft region 430A that is sized and shaped to fit within the vessel 12 and a proximal shaft region 430B that is positioned outside the patient and that is used to move the distal shaft region 430A in the vessel 12. Additionally, in one embodiment, the shaft 430 includes a first lumen 430C that defines at least a portion of the first passageway 434, and a second lumen 430D that defines the second passageway 436. Moreover, the shaft 430 can define a third lumen 430E (only partly illustrated in FIG. 5) that connects the expander 432 in fluid communication with a fluid pump (not shown).

The size, shape and materials used in the shaft 430 can be varied to suit the location of the treatment site 14. In one non-exclusive embodiment, the shaft 430 has an outer diameter of between approximately 1.0 mm and 2.5 mm; and a length of between approximately 100 cm and 150 cm. Further, the shaft 430 can be made of a flexible material such as polyurethane or other plastics. However, other diameters, lengths, or materials can be utilized.

The expander 432 is secured to the shaft 430 near the distal shaft region 430A. In one embodiment, the expander 432 is movable between a retracted position 432A and an expanded position (not shown) and includes a distal expander edge 432B and a proximal expander edge 432C. In the embodiment illustrated in FIGS. 4 and 5, the expander 432 is an inflatable balloon. With this design, a fluid pump can be used to control a fluid to selectively inflate the balloon to move the expander 432 from the retracted position 432A to the expanded position; and selectively deflate the balloon to move the expander 432 from the expanded position to the retracted position 432A. With this design, the expander 432 can be used to deploy a stent 18 or to perform an angioplasty procedure.

The size, shape and materials used in the expander 432 can be varied to suit the condition of the treatment site 14 and the desired movement characteristics of the expander 432. In one non-exclusive embodiment, the balloon has an outer diameter in the expanded position of between approximately 1.5 mm to 10 mm; and a length of between approximately 5 mm to 50 mm. Further, the balloon can be made of a flexible material such as polyurethane or other plastics. However, other diameters, lengths, or materials can be utilized.

Alternatively, the expander 432 can have a different design than a balloon. Still alternatively, the treatment device 16C can be used to deliver a self expanding stent. In this embodiment, the treatment device 16C does not need an expander 432.

The first passageway 434 extends through at least a portion of the shaft 430 and receives the first guide wire 16A. The first passageway 434 includes a first passageway inlet 434A that receives the first guide wire 16A and a first passageway outlet 434B. In one embodiment, the first passageway inlet 434A is positioned between the distal shaft region 430A and the proximal shaft region 430B. More specifically, the first passageway inlet 434A is positioned closer to the proximal expander edge 432C than the distal expander edge 432B. In the embodiment illustrated in FIG. 5, the first passageway 434 extends through a portion of the expander 432. In this embodiment, the first passageway inlet 434A is positioned in the expander 432. Further, in this embodiment, the first passageway outlet 434B is positioned near the proximal shaft region 430B.

With this design, the first guide wire 16A passes through the expander 432 proximal to the wider angle side of the first edge 222A of the stent 18. Stated in another fashion, the stent 18 can be positioned on the expander 432 so that the first guide wire 16A is positioned adjacent to the narrow portion of the triangular region 214B (illustrated in FIG. 2A) of the stent 18. Stated in yet another fashion, the stent 18 can be positioned on the expander 432 so that the first passageway inlet 434A is positioned adjacent to where the stent 18 is the shortest in length.

The second passageway 436 extends through the shaft 430 and receives the second guide wire 16B. The second passageway 436 includes a second passageway inlet 436A that receives the second guide wire 16B and a second passageway outlet 436B. In one embodiment, the second passageway inlet 436A is positioned at or near the distal shaft region 430A, and the second passageway outlet 436B is positioned near the proximal shaft region 430B.

It should be noted that when the treatment device 16C is positioned in the vessel 12 that the passageway inlets 434A, 436A are open to the blood in the vessel 12.

Additionally, FIG. 5 illustrates that the first guide wire 16A includes a first distal end 438A and a first proximal end 438B, and the second guide wire 16B includes a second distal end 440A and a second proximal end 440B.

Referring back to FIG. 4, the treatment device 16C ensures the proper alignment of the proximal stent edge 224A at the angled side branch 12B at the ostium 12C. The first guide wire 16A leaves the treatment device 16C at the site where the proximal portion of the stent 18 should be positioned at the distal part of the side branch ostium 12C. The second guide wire 16B exits the treatment device 16C distal to the stent 18. With this design, as the stent 18 is advanced into the coronary system on both wires 16A, 16B; it rotates automatically to position the stent 18 with the carina part exactly at the carina of the ostium 12C, which is ensured by the first guide wire 16A exiting the treatment device 16C at the proximal edge 222A of the stent 18 exactly at the site where it should be positioned. The narrow angle site will cover the proximal part of the ostium as long as the stent angle selected is close enough to the branch take off angle. The two guide wires 16A, 16B splitting away in a somewhat “Y” configuration ensure the stent 18 rotation and advancement exactly to (but not further) than the ostium 12C of the side branch by simple pushing (or gentle push and pull). To ensure the full coverage of the proximal part of the ostium 12C, the stent 18 selection should err on the side of more acute angle.

FIG. 6 illustrates an enlarged view of a portion of another treatment device 616C that is somewhat similar to the treatment device 16C illustrated in FIG. 5. However, in this embodiment, the first passageway inlet 634A is positioned in the shaft 630 prior to the expander 632. With this design, the first guide wire 16A exits the treatment device 616C prior to the expander 632.

With reference to all of the Figures, one, simplified, non-exclusive method for inserting the stent 18 includes the steps of: (i) inserting a sheath/guide catheter (not shown) into the vessel 12 in the groin or arm, (ii) taking an x-ray (fluoroscopy) on the patient to locate and evaluate the treatment site 14, (iii) inserting the guide wires 16A and 16B into the vessel 12 through the sheath/guide catheter and moving the second guide wire 16B past the treatment site 14 in the side branch 12B and inserting the first guide wire 16A into main vessel 12 past the ostium 12C of the side branch 12B, (iv) moving a balloon catheter (e.g. similar to the treatment device 16C) over the guide wires 16A, 16B and performing angioplasty on the treatment site 14, (iv) removing the balloon catheter, (v) moving a treatment device 16C with a stent 18 over the guide wires 16A and 16B to the treatment site 14, (vi) moving the stent 18 so that first edge 222A is parallel and aligned with ostium 12C which is achieved by simple pushing the stent 18 till resistance of the separating guide wires 16A, 16B is felt, (vii) inflating the treatment device 16B to move the stent 18 from the retracted configuration 218B to the expanded configuration 18A, and (viii) deflating treatment device 16B and removing the treatment device 16B (ix) the first guide wire 16A can be now used for the main branch stenting with classical stents/methods if the need be or removed from the vessel 12.

Further, while the particular stent 18 and treatment device 16C as shown and disclosed herein is fully capable of obtaining the objects and providing the advantages herein before stated, it is to be understood that it is merely illustrative of the presently preferred embodiments of the invention and that no limitations are intended to the details of construction or design herein shown other than as described in the appended claims.

What is claimed is:

1. A treatment device for treating a treatment site of a vessel of a mammal with the use of a first guide wire and a second guide wire, the treatment device comprising:
a tubular shaft having a distal shaft region that is sized and shaped to fit within the vessel and a proximal shaft region that is used to move the distal shaft region in the vessel;
a first passageway that extends along at least a portion of the shaft and that receives the first guide wire, the first passageway having a first passageway inlet that receives the first guide wire and a first passageway outlet, the first passageway inlet being positioned between the distal shaft region and the proximal shaft region, and the first passageway outlet being positioned near the proximal region; and
a second passageway that extends along the shaft and that receives the second guide wire, the second passageway having a second passageway inlet that receives the second guide wire and a second passageway outlet, the second passageway inlet being positioned at the distal shaft region, and the second passageway outlet being positioned near the proximal region.

2. The treatment device of claim 2 wherein the shaft includes a first lumen that defines at least a portion of the first passageway, and a second lumen that defines the second passageway.

3. The treatment device of claim 1 further comprising an expander that is secured to the shaft near the distal shaft region, the expander being movable between a retracted position and an expanded position, the expander including a distal expander edge and a proximal expander edge.

4. The treatment device of claim 3 wherein the first passageway inlet is positioned closer to the proximal expander edge than the distal expander edge.

5. The treatment device of claim 4 wherein the expander includes an inflatable balloon.

6. The treatment device of claim 4 wherein the first passageway extends through at least a portion of the expander.

7. The treatment device of claim 7 wherein the first passageway inlet is positioned in the expander.

8. The treatment device of claim 1 wherein the first passageway inlet is positioned in the shaft.

9. A balloon catheter for treating a treatment site of a vessel of a mammal with the use of a first guide wire and a second guide wire, the balloon catheter comprising:
a tubular shaft having a distal shaft region that is sized and shaped to fit within the vessel and a proximal shaft region that is used to move the distal shaft region in the vessel;
an inflatable balloon that is secured to the shaft near the distal shaft region, the balloon being movable between a retracted position and an expanded position, the balloon including a distal balloon edge and a proximal balloon edge;
a first passageway that extends along at least a portion of the shaft and that receives the first guide wire, the first passageway having a first passageway inlet that receives the first guide wire and a first passageway outlet, the first passageway inlet being positioned closer to the proximal balloon edge than the distal balloon edge; and
a second passageway that extends along the shaft and that receives the second guide wire, the second passageway having a second passageway inlet that receives the second guide wire and a second passageway outlet, the second passageway inlet being positioned at the distal shaft region, and the second passageway outlet being positioned near the proximal region

10. The balloon catheter of claim 9 wherein the shaft includes a first lumen that defines at least a portion of the first passageway, and a second lumen that defines the second passageway.

11. The balloon catheter of claim 9 wherein the first passageway extends through at least a portion of the balloon and wherein the first passageway inlet is positioned in the balloon.

12. The balloon catheter of claim 9 wherein the first passageway inlet is positioned in the shaft.

13. A method for treating a treatment site of a vessel of a mammal, the vessel including a main branch, and a side branch, the treatment site being located in the side branch near an intersection of the side branch and the main branch, the method comprising the steps of:
providing a first guide wire having a first distal end and a first proximal end;
moving the first distal end of the first guide wire in the main branch until the first distal end is past the intersection of the side branch with the first distal end still in the main branch;
providing a second guide wire having a second distal end and a second proximal end;
moving the second distal end of the second guide wire in the main branch and into side branch until the second distal end is past the treatment site;
providing a treatment device including (i) a tubular shaft having a distal shaft region that is sized and shaped to fit within the vessel and a proximal shaft region that is used to move the distal shaft region in the vessel; (ii) a first passageway that extends along at least a portion of the shaft and that receives the first guide wire, the first passageway having a first passageway inlet that receives the first guide wire and a first passageway outlet, the first passageway inlet being positioned between the distal shaft region and the proximal shaft region, and the first passageway outlet being positioned near the proximal region; and (iii) a second passageway that extends along the shaft and that receives the second guide wire, the second passageway having a second passageway inlet that receives the second guide wire and a second passageway outlet, the second passageway inlet being positioned at the distal shaft region, and the second passageway outlet being positioned near the proximal region; and
moving the treatment device over the guide wires until a portion of the treatment device is positioned adjacent to the treatment site.

14. The method of claim 13 wherein the treatment device includes an expander that is secured to the shaft near the distal shaft region, the expander being movable between a retracted position and an expanded position, the expander including a distal expander edge and a proximal expander edge; wherein the first passageway inlet is positioned closer to the proximal expander edge than the distal expander edge.

15. The method of claim 14 wherein the expander includes an inflatable balloon.

16. The method of claim 15 wherein the first passageway extends through at least a portion of the expander.

17. The method of claim 14 further comprising the step of positioning a stent on the expander.

18. The method of claim 13 wherein the first passageway inlet is positioned in the shaft.
